(19) United States
(12) Patent Application Publication
(10) Pub. No.: US 2025/0352597 A1
(43) Pub. Date: Nov. 20, 2025

(54) Hair Care Composition For Tender Scalps

(71) Applicant: David A Howard, New Orleans, LA (US)

(72) Inventor: David A Howard, New Orleans, LA (US)

(21) Appl. No.: 19/284,627

(22) Filed: Jul. 29, 2025

Related U.S. Application Data
(60) Provisional application No. 63/676,528, filed on Jul. 29, 2024.

(51) Int. Cl. A61K 36/53; A61K 8/34; A61K 8/35; A61K 8/368; A61K 8/42; A61K 8/73; A61K 8/92; A61K 8/9789; A61K 8/9794; A61K 8/99; A61K 9/00; A61K 31/05; A61K 31/085; A61K 31/125; A61K 31/167; A61K 31/192; A61K 36/61; A61K 47/10; A61K 47/32; A61K 47/44; A61K 47/46; A61Q 5/00

(52) U.S. Cl. CPC A61K 36/53; A61K 8/347; A61K 8/35; A61K 8/368; A61K 8/42; A61K 8/735; A61K 8/922; A61K 8/9789; A61K 8/9794; A61K 8/99; A61K 9/0014; A61K 31/05; A61K 31/085; A61K 31/125; A61K 31/167; A61K 31/192; A61K 36/61; A61K 47/10; A61K 47/32; A61K 47/44; A61K 47/46; A61Q 5/00; A61K 2800/30; A61K 2800/48

(57) ABSTRACT

A hair care composition is provided for alleviating scalp discomfort associated with hair manipulation procedures such as braiding, twisting, or detangling. The composition is offered in both non-medicated and medicated formulations, each comprising active analgesic agents suspended in a dermally compatible carrier. Non-medicated embodiments include Methyl Salicylate, Menthol, Eugenol, Camphor, and Capsaicin in a water-based base; medicated embodiments incorporate Lidocaine, Benzocaine, or Boronicaine with botanical adjuncts in an oil-based or aqueous carrier. The composition delivers rapid topical analgesia within 30 to 90 seconds of application and may be dispensed as a serum, spray, or roll-on. Pediatric and adult-specific formulations are disclosed. Embodiments address use during styling or post-styling for pain relief in textured-hair communities. Formulations are preservative-stabilized, hypoallergenic, and compatible with cultural grooming routines. Aseptic compounding, closed-vessel blending, and packaging options are described for dosage control and ingredient preservation.

### CROSS-REFERENCE TO RELATED APPLICATIONS

This application claims the benefit of priority of pending U.S. Provisional Application No. 63/676,528, filed Jul. 29, 2024, the disclosure of which is hereby incorporated by reference in its entirety.

### BACKGROUND OF THE INVENTION

### Field of the Invention

The present invention relates generally to pharmaceutical compositions for topical application, and more particularly to hair care compositions designed to alleviate scalp pain, discomfort, and hypersensitivity commonly associated with hair styling practices, such as braiding, twisting, combing, and detangling. The invention further pertains to both medicated and non-medicated formulations comprising botanical and pharmaceutical analgesic agents designed to provide safe, localized, and rapid relief upon topical administration to the scalp or adjacent dermal regions.

### Description of Related Art

Scalp pain and discomfort during and after hair styling procedures remain a widespread and under-addressed problem affecting diverse populations, particularly individuals with tightly coiled or textured hair types. Common grooming practices such as braiding, twisting, parting, and detangling frequently impose mechanical stress on the scalp, leading to localized pain, inflammation, and dermal hypersensitivity. In pediatric grooming contexts, the associated discomfort may elicit crying, resistance, or distress, thereby introducing emotional and psychological burdens for both children and caregivers.

Despite the prevalence of this issue, commercially available products typically fail to provide targeted relief during hair styling routines. Oral analgesics, although effective for general pain relief, are not suited for rapid, localized application and may carry undesirable systemic side effects, contraindications, or delayed onset times. Likewise, conventional topical analgesics are generally formulated for musculoskeletal or sports injuries and are not optimized for scalp use. Such preparations may contain high levels of alcohol, synthetic stabilizers, or occlusive agents that interfere with hair styling products or irritate the delicate scalp environment. Moreover, these products often leave visible residue, possess undesirable odors, or are incompatible with cultural styling practices requiring prolonged hairstyle retention.

There remains a significant unmet need for a fast-acting, residue-free, topically administered analgesic formulation that can be safely applied during or after hair styling without disrupting the hairstyle or interfering with products such as oils, gels, pomades, or styling foams. The need is particularly acute in underserved populations, including individuals with textured hair, pediatric users, and those who experience recurring discomfort following installation of protective styles, such as braids, locs, or sew-ins.

In recent years, consumer demand has increased for natural or plant-derived alternatives to conventional analgesics, including compositions free of parabens, phthalates, synthetic fragrances, or artificial dyes. Simultaneously, heightened awareness of skin sensitivities, allergic reactions, and dermatologic conditions such as seborrheic dermatitis, eczema, or post-inflammatory hyperreactivity have prompted a need for gentler formulations capable of providing therapeutic relief without exacerbating underlying conditions.

Accordingly, there exists a pronounced and growing need in the field of hair care and topical pharmaceuticals for compositions that deliver targeted analgesia, are safe for repeated topical use, and are available in both medicated and non-medicated forms. These compositions must be adaptable for use in varied delivery systems—such as roll-ons, sprays, serums, or creams—and formulated with consideration for pediatric users, fragrance-sensitive individuals, and the cultural grooming needs of textured-hair communities.

### SUMMARY OF THE INVENTION

The following presents a simplified summary of the present disclosure in a simplified form as a prelude to the more detailed description that is presented herein.

Therefore, in accordance with embodiments of the invention, there is provided a hair care composition formulated to alleviate scalp discomfort, pain, and tenderness during or after hair manipulation procedures. In one embodiment, the hair care composition comprises a non-medicated formulation including approximately 2.0% by weight Eugenol, approximately 1.0% by weight Menthol, approximately 3.15% by weight Methyl Salicylate, approximately 0.50% by weight Lavender Oil, approximately 0.50% by weight Peppermint Oil, and approximately 0.10% by weight Capsaicin. This formulation is preferably suspended in a base of approximately 79.30% by weight Distilled Water, 2.0% by weight Glycerin, 0.75% by weight Hyaluronic Acid, 1.0% by weight Lecithin, and 2.0% by weight Jojoba Oil, with optional inclusion of approximately 0.50% by weight Tea Tree Oil. The formulation may be preserved using approximately 4.0% by weight Leucidal SF Complete and may include approximately 1.0% by weight Skullcap Extract to calm irritated skin.

In another embodiment, the hair care composition is a fragrance-free and allergen-reduced formulation comprising approximately 2.0% by weight Eugenol, approximately 1.0% by weight Menthol, approximately 3.15% by weight Methyl Salicylate, and approximately 0.10% by weight Capsaicin, excluding Lavender Oil, Peppermint Oil, and Tea Tree Oil. In another embodiment, the non-medicated formulation includes the above actives along with approximately 3.0% by weight Camphor, structured with approximately 0.20% by weight Carbomer and 2.0% by weight GelMaker NAT. In another embodiment, the non-medicated formulation is delivered as a post-braid relief spray.

In another embodiment, a medicated hair care composition is provided for more intense scalp pain, including approximately 1.2% by weight Lidocaine Hydrochloride, 3.0% by weight Eugenol, and 1.0% by weight Menthol, with the balance being Jojoba Oil. In a pediatric embodiment, the composition includes approximately 0.6% by weight Lidocaine HCL, 2.0% by weight Eugenol, and 0.5% by weight Menthol. In another embodiment, a medicated composition includes a therapeutically effective amount of one or more anesthetic agents selected from Lidocaine, Lidocaine HCL, Benzocaine, or Boronicaine, optionally in combination with botanical analgesics such as Eugenol, Menthol, Methyl Salicylate, and Capsaicin, suspended in Jojoba Oil or an aqueous carrier. In another embodiment, the medicated composition further comprises CBD at concentrations ranging from 0.1% to 1.0% by weight.

In another embodiment, the hair care composition is delivered in a form selected from a serum, gel, spray, roll-on, single-use applicator, or twist-cap ampoule. In another embodiment, the composition is prepared via aseptic closed-vessel blending. In yet another embodiment, the composition is formulated for use following hair removal procedures, including waxing or shaving, to reduce irritation.

In one embodiment, the therapeutically effective amount reduces perceived scalp pain by at least 30% within approximately 30 to 90 seconds of application. In another embodiment, the composition provides alternating warm and cool sensations due to the interaction of Methyl Salicylate, Menthol, and Capsaicin. In another embodiment, Lidocaine is used in place of Methyl Salicylate in medicated compositions for users who are sensitive to salicylates or require pharmaceutical-grade anesthetics.

The embodiments disclosed herein address a longstanding unmet need, especially among textured-hair communities and pediatric users, by delivering targeted pain relief, minimizing crying during grooming, and promoting a culturally resonant and emotionally supportive styling experience.

### DETAILED DESCRIPTION

Referring now to the present disclosure, hair care compositions are described that are specially formulated to alleviate scalp discomfort, pain, and tenderness associated with hair manipulation procedures such as braiding, twisting, combing, parting, or detangling. The disclosed hair care compositions are provided in both a medicated formulation as well as a non-medicated formulation, each comprising active and inactive ingredients configured to deliver targeted analgesic effects upon topical application to the scalp. The hair care compositions are formulated for application during hair styling procedures, such as braiding, twisting, or parting, to deliver immediate and localized pain relief without interrupting or interfering with the styling process.

In one embodiment, a hair care composition comprises a non-medicated formulation including approximately 2.0% by weight Eugenol, approximately 1.0% by weight Menthol, approximately 3.15% by weight Methyl Salicylate, approximately 0.50% by weight Lavender Oil, approximately 0.50% by weight Peppermint Oil (Mentha Piperita Oil), and approximately 0.10% by weight Capsaicin. Each of these active ingredients may be dissolved or suspended in a base comprising approximately 79.30% by weight Distilled Water, approximately 2.0% by weight Glycerin, approximately 0.75% by weight Hyaluronic Acid, approximately 1.0% by weight Lecithin, and approximately 2.0% by weight Jojoba Oil. The non-medicated hair care composition may be preserved using approximately 4.0% by weight Leucidal SF Complete (Leuconostoc/Radish Root Ferment Filtrate) and may further comprise approximately 1.0% by weight Skullcap Extract to calm irritated skin. The non-medicated formulation may optionally include approximately 0.50% by weight Tea Tree Oil (Melaleuca Alternifolia Leaf Oil). In another embodiment, the non-medicated hair care composition is provided in a fragrance-free formulation configured for users with heightened skin sensitivity or allergic response to fragrance components.

In another embodiment, a non-medicated hair care composition is formulated specifically for users with sensitive skin or heightened dermatological reactivity. This embodiment excludes common allergens, artificial dyes, and all fragrance components. The fragrance component is preferably vanilla almond. In preferred embodiments, ingredients such as Peppermint Oil, Tea Tree Oil, and Lavender Oil are excluded, while retaining approximately 2.0% by weight Eugenol, approximately 1.0% by weight Menthol, approximately 3.15% by weight Methyl Salicylate, and approximately 0.10% by weight Capsaicin. These active ingredients may be suspended in a base comprising approximately 79.30% by weight Distilled Water, approximately 2.0% by weight Glycerin, approximately 0.75% by weight Hyaluronic Acid, approximately 1.0% by weight Lecithin, and approximately 2.0% by weight Jojoba Oil. In one embodiment, the composition is fragrance-free and allergen-reduced formulation is targeted for individuals prone to flare-ups, irritation, or post-inflammatory sensitivity due to prior scalp conditions or dermatological therapies.

In this context, a “therapeutically effective amount” refers to an amount sufficient to reduce pain perception, scalp tenderness, or discomfort within approximately 30 to 90 seconds after application. For example, the concentration of Menthol may range from about 0.3% to about 1.0% by weight, Eugenol from about 1.5% to about 3.0% by weight, Methyl Salicylate from about 1.0% to about 5.15% by weight, Camphor from about 1.0% to about 5.0% by weight, and Capsaicin from about 0.001% to about 0.30% by weight. These concentrations have been observed to yield a soothing and desensitizing effect on tender scalp regions during hair manipulation.

The non-medicated hair care composition may be provided in two embodiments. In a first preferred embodiment, the non-medicated hair care composition includes approximately 2.0% by weight Eugenol, approximately 1.0% by weight Menthol, approximately 3.15% by weight Methyl Salicylate, approximately 0.50% by weight Lavender Oil, approximately 0.50% by weight Peppermint Oil, approximately 0.10% by weight Capsaicin, and optionally Tea Tree Oil, but excludes Camphor to accommodate users who are sensitive to warming sensations. In a second preferred embodiment, the non-medicated hair care composition includes the same ingredients as the first embodiment and further includes Camphor at approximately 3.0% by weight to enhance the warming and analgesic sensation. Both embodiments are thickened using approximately 0.20% by weight Carbomer and structured using approximately 2.0% by weight GelMaker NAT, which may include Sodium Acrylate/Sodium Acryloyldimethyl Taurate Copolymer, Polyglyceryl-6 Laurate, and Polyglycerin-6. The non-medicated hair care compositions may optionally include approximately 0.50% by weight of a mild fragrance. The mild fragrance is preferably vanilla almond. The non-medicated hair care compositions achieve an analgesic effect for users seeking natural scalp pain relief.

In another embodiment, the hair care compositions are formulated for use beyond hair styling scenarios, including applications following hair removal procedures such as waxing, shaving, eyebrow grooming, or depilation in the leg or genital regions. These formulations may include either medicated or non-medicated versions and are applied post-procedurally to reduce irritation, redness, or stinging sensation in the affected dermal areas. The ability of the active ingredients to deliver prompt topical relief enhances user comfort during routine grooming and supports a wider therapeutic application profile.

In another embodiment, a hair care composition comprises a medicated formulation for more acute or intense scalp pain. In one embodiment, the medicated hair care composition includes Lidocaine or Lidocaine Hydrochloride (HCL), Eugenol, and

Menthol dissolved in Jojoba Oil. In a pediatric embodiment, the medicated hair care composition comprises approximately 0.6% by weight Lidocaine HCL, about 2.0% by weight Eugenol, and about 0.5% by weight Menthol, with the balance being Jojoba Oil by weight. In an adult embodiment, the medicated hair care composition comprises approximately 1.2% by weight Lidocaine HCL, about 3.0% by weight Eugenol, and about 1.0% by weight Menthol, with the balance being Jojoba Oil by weight. These anesthetic agents work by blocking pain signals from the nerves. Each embodiment is optimized for safe and effective use in its intended demographic. Users are cautioned that dilution of the medicated hair care compositions may reduce analgesic effectiveness.

In another embodiment, the interaction between Methyl Salicylate, Menthol, and Capsaicin is selected to provide a synergistic analgesic effect through alternating warm and cool sensations. Methyl Salicylate acts as a rubefacient to stimulate blood flow, Menthol provides a cooling counter-irritant sensation, and Capsaicin activates TRPV1 receptors to desensitize peripheral nerve endings. This combination promotes rapid sensory adaptation to pain and enhances the composition's therapeutic efficacy when applied to tender scalp regions.

In another embodiment, the medicated hair care composition is prepared in a water-based carrier instead of an oil-based carrier. In this embodiment, the active ingredients—such as Lidocaine, Lidocaine Hydrochloride (HCL), Benzocaine, or Boronicaine, in combination with botanical analgesics including Eugenol, Menthol, and optionally Methyl Salicylate or Capsaicin—are solubilized or suspended in an aqueous base comprising Distilled Water, Glycerin, Lecithin, and Hyaluronic Acid. This water-based formulation is particularly suited for users who prefer a lighter consistency or for use immediately before or after heat-based styling procedures, as it evaporates more quickly than oil-based alternatives. The water-based carrier enables faster absorption and is suitable for users with oily scalp types or who require a formulation that does not interfere with styling oils.

In another embodiment, substitution of Lidocaine or Lidocaine HCL for Methyl Salicylate is preferred in medicated hair care compositions. This substitution ensures compliance with over-the-counter topical analgesic regulations and reduces the potential for dermal irritation in sensitive users. The substitution is particularly useful in pediatric or elderly patients, where enhanced safety margins are desired. Lidocaine-based formulations are especially advantageous when rapid numbing is required without relying on salicylate esters.

In yet another embodiment, a medicated hair care composition comprises a therapeutically effective amount of one or more anesthetic agents selected from Lidocaine, Lidocaine Hydrochloride (HCL), Benzocaine, or Boronicaine, in combination with one or more botanical analgesics selected from Eugenol, Menthol, Methyl Salicylate, and (optionally) preferably Capsaicin. This embodiment may further include Cannabidiol (CBD) as an optional ingredient to enhance soothing and anti-inflammatory properties. The active ingredients are preferably dissolved or suspended in a dermally acceptable carrier such as Jojoba Oil. The concentration of anesthetic agents may range from 0.5% to 4.0% by weight, and botanical components from 1.0% to 5.0% by weight. CBD, when included, may be present at concentrations ranging from 0.1% to 1.0% by weight. The composition is preferably formulated to deliver fast-acting, targeted relief when applied directly to tender or inflamed scalp regions during or following hair styling procedures. This embodiment is suited for individuals experiencing elevated scalp sensitivity and may be delivered in the form of a serum, roll-on, topical oil, or spray.

These medicated hair care compositions may be prepared under aseptic compounding protocols, with ingredients weighed and dissolved sequentially while maintaining a closed-system blending process. During preparation, magnetic stirring bars may be employed under sterile conditions. Final products are filtered, packaged in opaque or UV-resistant containers, and labeled in compliance with FDA topical analgesic guidance. Pharmacist instructions include verification of solubilization before filtration and storage at controlled room temperature.

The carrier in the medicated hair care compositions consists primarily of Jojoba Oil, selected for its compatibility with sensitive skin and minimal interference with hair styling products. This carrier enhances dermal absorption of the anesthetic agents while maintaining a clean, non-greasy finish. The medicated formulation may optionally contain antioxidant stabilizers or dermal conditioners to preserve ingredient efficacy.

In another embodiment, the hair care composition is provided in a pre-metered, single-use applicator configured for hygienic, targeted application to discrete scalp regions. The applicator may include a sterile, break-seal ampoule, twist-cap reservoir, or sponge-tipped applicator to facilitate precise dosing without direct contact. In other embodiments, the hair care composition may be dispensed via an integrated nozzle pump, calibrated roll-on dispenser, or aerosolized spray bottle configured to deliver a uniform distribution pattern. Packaging materials are preferably UV-stable and chemically resistant to preserve ingredient stability and formulation integrity throughout shelf life.

The hair care compositions can be delivered in various forms including a liquid, gel, cream, aerosol, spray, or roll-on. This enables users to target specific scalp zones such as the crown, hairline, nape, or parts where tension is greatest. In a preferred form, the hair care composition is provided as a lightweight serum that spreads evenly and dries quickly without leaving visible residue or disrupting hair styling products.

In another embodiment, the hair care compositions are formulated for use as post-styling or post-protective-style recovery treatments, including as a post-braid relief spray. These embodiments are applied immediately following hair styling procedures such as braiding, twisting, or sew-ins, where mechanical manipulation induces tension and discomfort. The composition may be dispensed in a fine mist or cooling spray directed to the hairline, crown, or parts subjected to sustained tension. The rapid analgesic action provides relief from tender head pain while promoting continued wear of the hairstyle without the need for early takedown or re-styling.

In another embodiment, the disclosed hair care compositions are formulated with emotional and cultural considerations for use in textured-hair communities. Pediatric versions reduce crying and discomfort during hair manipulation, thereby fostering a more pleasant grooming experience and improving parent-child bonding. These embodiments address a longstanding unmet need for individuals, especially children, experiencing heightened scalp sensitivity or “tender-headedness,” and provide targeted relief that aligns with culturally significant grooming practices.

The embodiments disclosed herein present many advantages, including the ability to relieve pain from tight hairstyles without resorting to oral medication. By targeting the scalp directly, the hair care compositions provide immediate localized pain relief, enhance user comfort, and permit uninterrupted hair styling. The use of natural analgesics such as Eugenol and Methyl Salicylate minimizes dependency on synthetic pharmaceuticals while maintaining effectiveness. Pediatric-specific and adult-specific medicated hair care compositions further tailor the product's effectiveness and safety to diverse users, including tender-headed children undergoing hair manipulation procedures. Moreover, the disclosed formulations fill a longstanding market gap by addressing tender head pain and related scalp discomfort not adequately treated by traditional analgesics.

Except as may be expressly otherwise indicated, the article “a” or “an” if and as used herein is not intended to limit the meaning to a singular form of the item but encompasses multiple equivalents as well.

The spirit and scope of the appended claims should not be limited to the description of the preferred versions contained herein but should be interpreted broadly and in accordance with the full breadth of the claims and their legal equivalents.

1. A hair care composition comprising:
a therapeutically effective amount of Eugenol;
a therapeutically effective amount of Menthol;
a therapeutically effective amount of Methyl Salicylate;
a therapeutically effective amount of Camphor;
a therapeutically effective amount of Peppermint Oil;
a therapeutically effective amount of Lavender Oil;
a carrier comprising Jojoba Oil; and
wherein the composition is formulated for topical application to the scalp to reduce pain associated with hair styling.

2. The hair care composition of claim 1, further comprising Capsaicin in an amount sufficient to produce a warming sensation upon application to the scalp.

3. The hair care composition of claim 1, wherein the composition comprises Leucidal SF Complete to aid dermal absorption.

4. The hair care composition of claim 1, wherein the composition further comprises Hyaluronic Acid to provide hydration to the scalp.

5. The hair care composition of claim 1, wherein the composition further comprises Skullcap Extract to support calming of irritated skin.

6. The hair care composition of claim 1, wherein the composition is in a water-based serum form comprising Carbomer and GelMaker NAT as thickening agents.

7. The hair care composition of claim 1, wherein the composition is residue-free, safe for daily use, and compatible with any hair type and styling products.

8. A medicated hair care composition comprising:
a therapeutically effective amount of Lidocaine or Lidocaine Hydrochloride (HCL);
a therapeutically effective amount of Eugenol;
a therapeutically effective amount of Menthol;
Jojoba Oil as a carrier; and
wherein the composition is formulated for topical application to the scalp to provide analgesic relief during hair styling procedures.

9. The medicated hair care composition of claim 8, wherein the composition comprises between 0.5% and 3% by weight Eugenol.

10. The medicated hair care composition of claim 8, wherein the composition comprises between 0.3% and 1% by weight Menthol.

11. The medicated hair care composition of claim 8, wherein the composition comprises between 0.5% and 1.5% by weight Lidocaine or Lidocaine HCL.

12. The medicated hair care composition of claim 8, wherein the composition is formulated specifically for children or adults, with dosages adjusted accordingly.

13. The medicated hair care composition of claim 8, wherein the composition may alternatively comprise Benzocaine or Boronicaine in place of Lidocaine.

14. The medicated hair care composition of claim 8, wherein the formulation is provided in liquid, gel, cream, aerosol, or spray form for direct scalp application.

15. A hair care composition for relieving tender head pain during hair manipulation, comprising:
a combination of natural analgesic agents including Eugenol, Menthol, Clove Leaf Oil, and Methyl Salicylate;
a stimulation agent selected from the group consisting of Capsaicin, Camphor, or both;
a carrier system including Distilled Water, Glycerin, and Jojoba Oil; and
an emulsifier-thickener complex including Carbomer and GelMaker NAT,
wherein the composition is adapted to desensitize the scalp and mitigate pain sensitivity during hair detangling, braiding, twisting, or styling.

16. The hair care composition of claim 15, wherein the composition comprises Lavender Oil and Peppermint Oil to provide a cooling and soothing effect.

17. The hair care composition of claim 15, wherein the composition includes Leucidal SF Complete as a natural preservative and dermal penetration enhancer.

18. The hair care composition of claim 15, wherein the composition includes fragrance and is free of alcohol, parabens, and synthetic colorants.

19. The hair care composition of claim 15, wherein the composition achieves at least a 60% reduction in pain within 60 seconds of topical application.

20. The hair care composition of claim 15, wherein the composition is specifically designed for individuals with textured hair or high scalp sensitivity.

21. The hair care composition of claim 15, wherein the composition is residue-free and suitable for use during or after hair manipulation routines.
